(19) United States
(12) Patent Application Publication
(10) Pub. No.: US 2025/0229248 A1
(43) Pub. Date: Jul. 17, 2025

(54) CONTINUOUS-FLOW PREPARATION METHOD OF AMINO ALCOHOL COMPOUNDS

(71) Applicants: Fudan University, Shanghai (CN); Jiangxi Normal University, Nanchang (CN)

(72) Inventors: Fener CHEN, Shanghai (CN); Xianjing ZHENG, Shanghai (CN); Chang LIU, Shanghai (CN); Yiqun TIAN, Shanghai (CN); Yajiao ZHANG, Shanghai (CN); Li WAN, Shanghai (CN); Chunxue PENG, Shanghai (CN); Songlin ZHENG, Shanghai (CN); Liangchuan LAI, Shanghai (CN)

(21) Appl. No.: 19/098,176

(22) Filed: Apr. 2, 2025

(30) Foreign Application Priority Data
Apr. 23, 2024 (CN) 202410488186.3

(51) Int. Cl. B01J 19/00; C07C 213/08

(52) U.S. Cl. CPC B01J 19/0093; C07C 213/08; B01J 2219/00792; B01J 2219/0086; B01J 2219/00873; B01J 2219/00889; B01J 2219/00891; B01J 2219/00905; B01J 2219/00959; B01J 2219/00961; B01J 2219/00963; B01J 2219/00984

(57) ABSTRACT

A continuous-flow preparation method for an amino alcohol compound using a micro-reaction system. The micro-reaction system includes a feed pump, a micromixer, a microchannel reactor, and a back pressure valve. An aldehyde compound and an amine compound are simultaneously fed to the micro-mixer for mixing to obtain a mixed solution. The mixed solution is directly fed to the micro-channel reactor and undergoes an addition reaction. The reaction mixture is collected, and subjected to concentration, separation and purification to obtain the desired amino alcohol compound.

### CROSS-REFERENCE TO RELATED APPLICATIONS

This application claims the benefit of priority from Chinese Patent Application No. 202410488186.3, filed on Apr. 23, 2024. The content of the aforementioned application, including any intervening amendments thereto, is incorporated herein by reference in its entirety.

### TECHNICAL FIELD

This application relates to pharmaceutical engineering, and more specifically to a method of preparing amino alcohols.

### BACKGROUND

Amino alcohols are a class of compounds with unique chemical structures, containing both amino groups (NH₂) and hydroxyl groups (OH). Due to their diverse molecular structures, amino alcohols have a wide range of applications in various fields. They play a significant role in the pharmaceutical synthesis, organic synthesis, chemical catalysis, and other chemical processes. Some typical amino alcohols include ethanolamine, propanolamine, and isopropanolamine, which are used in the preparation of chemical products, such as pharmaceuticals, coatings, adhesives, surfactants and pigments.

The general chemical structural formula of the amino alcohol is as follows:

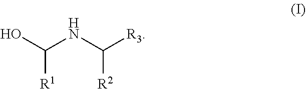

Currently, many methods have been developed for synthesizing amino alcohols. Among them, the most commonly used method involves the hydrogenation of hydroxypropionitrile in the presence of ammonia using a modified Raney nickel catalyst. This method features a relatively short process and high atom economy (95%). However, it also faces several challenges, such as difficult technical operation and separation and recovery of the Raney nickel catalyst. Li Baoqiang et al. (Preparation of 5-Amino-1-pentanol, Fine Chemical Intermediates, 2014, 6, 40-42) develops another synthesis method, in which 1,5-pentanediol as the starting material undergoes monochlorination reaction with concentrated hydrochloric acid to produce 5-chloropentanol, which is then further reacted with ammonia gas to yield the amino alcohol through amination. The product can be purified to reach a purity over 99% via distillation. However, this method is less efficient and generates a significant amount of waste. Chinese Patent Publication No. 112469692A discloses a method for synthesizing amino alcohols through oxidation of amino compounds. For example, the corresponding amino alcohol can be synthesized through the oxidation of aminomethane with hydrogen peroxide, with water (H₂O) as a byproduct. In addition to their respective limitations, these methods also share common drawbacks, such as long reaction times in traditional batch reactors, cumbersome operations, serious safety risks, low efficiency, and high energy consumption, all of which hinder the large-scale production of amino alcohols.

### SUMMARY

An objective of the present disclosure is to provide a continuous-flow preparation method for amino alcohol compounds, which has high safety, short reaction time, low energy consumption, and high efficiency.

Compared to the existing preparation methods, this method has significantly enhanced safety, reduced energy consumption, shortened reaction time, and improved automation level and efficiency, and is thus well-suited for industrial applications.

Technical solutions of the present disclosure are described below.

A continuous-flow preparation method for an amino alcohol compound using a micro-reaction system, the micro-reaction system comprising a first feed pump, a second feed pump, a micromixer, a microchannel reactor and a back pressure valve, the micromixer, the microchannel reactor and the back pressure valve being connected in sequence, and the continuous-flow preparation method comprising:
(1) simultaneously feeding a solution of an aldehyde compound in a first solvent and a solution of an amine compound in a second solvent to the micromixer respectively through the first feed pump and the second feed pump for mixing to obtain a mixed

R¹—CHO

solution, wherein the aldehyde compound is represented by (II), and the amine compound is represented by

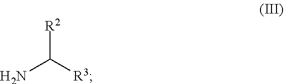

(2) feeding the mixed solution into the microchannel reactor to carry out an addition reaction to obtain a reaction mixture; and
(3) collecting the reaction mixture flowing out of the microchannel reactor, followed by concentration and separation purification to obtain the amino alcohol compound of formula (I):

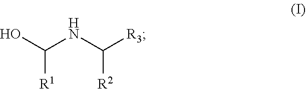

wherein R¹, R², and R³ are each independently selected from the group consisting of hydrogen, halogen, a C₁-C₁₂ alkyl, a C₃-C₆ cycloalkyl, a C₁-C₆ alkoxy, an unsaturated alkyl, an aromatic group, a nitrogen-containing alkyl, a sulfur-containing alkyl, a carboxyl group, an amide group, an aldehyde group, and an ester group.

In an embodiment, in step (1), the first solvent and the second solvent are each independently selected from the group consisting of toluene, ethylbenzene, acetonitrile, n-butyronitrile, acetone, butanone, methyl isobutyl ketone, tetrahydrofuran, 2-methyltetrahydrofuran, 1,4-dioxane, dimethylsulfoxide (DMSO), dimethylformamide (DMF), methanol, ethanol, n-propanol, isopropanol, n-butanol, isobutanol, ethyl ether, methyl tert-butyl ether, and a combination thereof. Preferably, the first solvent and the second solvent are each independently selected from the group consisting of acetone, butanone, methyl isobutyl ketone, tetrahydrofuran, 2-methyltetrahydrofuran, methanol, ethanol, n-propanol, isopropanol, n-butanol, isobutanol, ethyl ether, and methyl tert-butyl ether.

In an embodiment, in step (1), in step (1), a temperature in the micromixer is controlled at 25-60° C.; and flow rates of the solution of the aldehyde compound and the solution of the amine compound are controlled such that a molar ratio of the aldehyde compound to the amine compound in the micromixer is 1:0.5-6.

In an embodiment, in step (2), a temperature in the microchannel reactor is controlled at 30-60° C., preferably, 40-60° C.; and a residence time of the mixed solution in the microchannel reactor is 2-10 min, preferably, 5-10 min.

In an embodiment, in step (2), a back pressure of the back pressure valve is set to 0.1-2 MPa, preferably, 0.3-1.5 MPa.

In an embodiment, the micromixer is selected from the group consisting of a static mixer, a coaxial flow micromixer, a flow-focusing micromixer, a T-shaped mixer, a Y-shaped mixer, a Z-shaped mixer, a X-shaped mixer, a SK-type mixer, a SX-type mixer, a SV-type mixer, and a gourd-shaped divergent-convergent mixer.

In an embodiment, the micromixer is a circular-embedded square divergent-convergent mixer, which is shown in FIG. 2. Specifically, the micromixer is composed of a plurality (such as 4-10) of mixing units connected in series; each of the plurality of mixing unit comprises an outer square tube, an inner round tube, and an annular mixing channel arranged between the outer square tube and inner round tube; adjacent two mixing units among the plurality of mixing units are connected via a conduit at two opposite corners of outer square tubes of the adjacent two mixing units; and the mixed solution is divided in the annular mixing channel and converges at corners.

In an embodiment, the microchannel reactor is a tubular microchannel reactor or a plate-type microchannel reactor;
an inner diameter of the tubular microchannel reactor is 100 μm-8 mm, preferably, 100 μm-5 mm; and
the plate-type microchannel reactor comprises a first heat exchange layer, a reaction layer, and a second heat exchange layer sequentially arranged from top to bottom; and the reaction layer is provided with a reaction fluid channel having a hydraulic diameter of 100 μm-8 mm, preferably, 100 μm-5 mm.

In an embodiment, the microchannel reactor is a divergent-convergent mixer, which is shown in FIG. 3; the microchannel reactor is composed of a plurality (such as 10-20) of circular or elliptical rings connected sequentially; adjacent two circular or elliptical rings among the plurality of circular or elliptical rings are communicated via conduits, or are tangent to each other and communicated at a tangent point; and the adjacent two circular or elliptical rings form a gourd-shaped structure, such that the reaction mixture is divided within each of the adjacent two circular or elliptical rings and converge at junctions between the adjacent two circular or elliptical rings.

The present disclosure utilizes a micro-reaction system comprising a micromixer, a microchannel reactor, and a back-pressure device connected in sequence to carry out the addition reaction of aldehydes and amines for the preparation of amino alcohol compounds. Compared to traditional synthesis methods, it offers the following advantages.
(1) The multiphase mixing, mass transfer, and reaction processes are completed within the micromixer and microchannel reactor, which has simple operations, low equipment requirements and a high degree of automation, significantly reducing energy consumption and production costs while shortening reaction time;
(2) The addition reaction is carried out in the microchannel reactor, providing excellent atom utilization, good reaction reproducibility, and ease of scale-up.
(3) The use of highly toxic and carcinogenic reagents is avoided.

### BRIEF DESCRIPTION OF THE DRAWINGS

FIG. 1 is a schematic diagram of a micro-reaction system according to an embodiment of the present disclosure;

FIG. 2 is a schematic diagram of a gourd-shaped divergent-convergent micromixer according to an embodiment of the present disclosure; and

FIG. 3 is a schematic diagram of a gourd-shaped divergent-convergent microchannel reactor according to an embodiment of the present disclosure.

In the drawings:
1, feed pump; 2, oil bath; 3, micromixer; 4, microchannel reactor; 5, back pressure valve; 6, storage tank; 7, inlet channel of the micromixer; 8, outlet channel of the micromixer; 9, inlet channel of the microchannel reactor; and 10, outlet channel of the microchannel reactor.

### DETAILED DESCRIPTION OF EMBODIMENTS

The present disclosure will be described in further detail below with reference to embodiments.

### Example 1

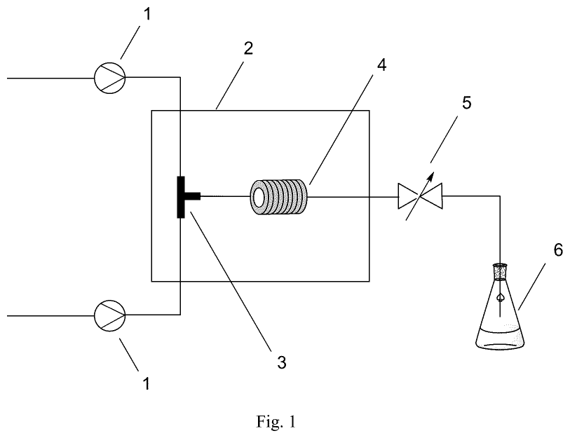

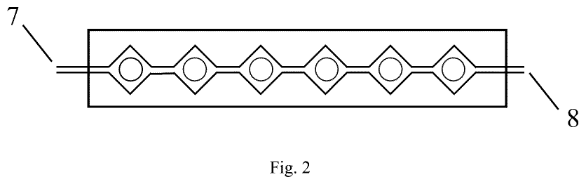

Provided herein was a method for continuous preparation of amino alcohol compounds using a micro-reaction system. As shown in FIG. 1, the micro-reaction system included a micromixer 3 and a microchannel reactor 4 sequentially connected to each other. The micro-reaction system further included two feed pumps 1, an oil bath 2, the micromixer 3, the microchannel reactor 4, a back pressure valve 5, and a storage tank 6. The feed pumps 1 were configured to regulate and control the flow rates of the reactants within the micro-reaction system. The oil bath 2 was configured to adjust and maintain the reaction temperature in the microchannel reactor 4. The back pressure valve 5 was configured to regulate and control the reaction pressure in the micro-reaction system. The storage tank 6 was configured to collect the reaction mixture. The micromixer 3 was a divergent-convergent micromixer (with structural details shown in FIG. 2) with an outer square tube and an inner round tube, where an inlet channel 7 and an outlet channel 8 of the micromixer 3 each had an inner diameter of 500 μm, a side length of the outer square tube was 5 mm, a diameter of the inner round tube was 4.5 mm, and the outer square tube and the inner round tube were concentric. A spacing between adjacent two square tubes was 12 mm. The microchannel reactor 4 was a polytetrafluoroethylene (PTFE) tubular microchannel reactor with an inner diameter of 0.8 mm and a reaction volume of 5 mL. The back pressure valve 5 was set to provide a pressure of 0.5 MPa. The preparation method was performed as follows.
(1) Paraformaldehyde (82.08 g, 2.0 eq) was added to 130 mL of methanol and stirred to dissolve to obtain solution 1. Alanine (40.6 g, 1.0 eq) was added to 200 mL of methanol and stirred to obtain solution 2.
(2) The flow rates of solution 1 and solution 3 were controlled to be 1.0 mL/min and 1.2 mL/min, respectively. The temperature in the micromixer was 45° C., and the temperature in the microchannel reactor was 45° C. The residence time for the reaction was 2.3 min.
(3) The reaction mixture flowing from the micro-reaction system was collected and subjected to concentration and separation and purification to obtain hydroxymethylalanine with a yield of 82%.

### Example 2

Provided herein was a method for continuous preparation of amino alcohol compounds using a micro-reaction system. The method was basically the same as that of Example 1, except that the solution 1 in this example was prepared by dissolving paraformaldehyde into the methyl tertiary butyl ether.

Specifically, the method provided herein included the following steps.
(1) Paraformaldehyde (82.08 g, 2.0 eq) was added to 130 mL of methyl tertiary butyl ether and stirred to dissolve to obtain solution 1. Alanine (40.6 g, 1.0 eq) was added to 200 mL of methanol and stirred to obtain solution 2.
(2) The flow rates of solution 1 and solution 2 were controlled to be 1.0 mL/min and 1.2 mL/min, respectively. The temperature in the micromixer was 45° C., and the temperature in the microchannel reactor was 45° C. The residence time for the reaction mixture was 2.3 min.
(3) The reaction mixture flowing from the micro-reaction system was collected and subjected to concentration and separation and purification to obtain hydroxymethylalanine with a yield of 70%.

### Example 3

Provided herein was a method for continuous preparation of amino alcohol compounds using a micro-reaction system. The method was basically the same as that of Example 1, except the temperature in the micromixer and the microchannel reactor.

Specifically, the method provided herein included the following steps.
(1) Paraformaldehyde (82.08 g, 2.0 eq) was added to 130 mL of methanol and stirred to dissolve to obtain solution 1. Alanine (40.6 g, 1.0 eq) was added to 200 mL of methanol and stirred to obtain solution 2.
(2) The flow rates of solution 1 and solution 2 were controlled to be 1.0 mL/min and 1.2 mL/min, respectively. The temperature in the micromixer was 60° C., and the temperature in the microchannel reactor was 60° C. The residence time for the reaction mixture was 2.3 min.
(3) The reaction mixture flowing from the micro-reaction system was collected and subjected to concentration and separation and purification to obtain hydroxymethylalanine with a yield of 89%.

### Example 4

Provided herein was a method for continuous preparation of amino alcohol compounds using a micro-reaction system. The method was basically the same as that of Example 1, except the composition of solutions 1 and 2 and the temperature in the micromixer and the microchannel reactor.

Specifically, the method provided herein included the following steps.
(1) Acetaldehyde (82.08 g, 2.0 eq) was added to 130 mL of methanol and stirred to dissolve to obtain solution 1. Methylamine (42.45 g, 1.0 eq) was added to 200 mL of methanol and stirred to obtain solution 2.
(2) The flow rates of solution 1 and solution 2 were controlled to be 1.0 mL/min and 1.2 mL/min, respectively. The temperature in the micromixer was 60° C., and the temperature in the microchannel reactor was 60° C. The residence time for the reaction mixture was 2.3 min.
(3) The reaction mixture flowing from the micro-reaction system was collected and subjected to concentration and separation and purification to obtain isopropanolamine with a yield of 82%.

### Example 5

Provided herein was a method for continuous preparation of amino alcohol compounds using a micro-reaction system. The method was basically the same as that of Example 1, except the composition of solutions 1 and 2 and the temperature in the micromixer and the microchannel reactor.

Specifically, the method provided herein included the following steps.
(1) Acetaldehyde (82.08 g, 2.0 eq) was added to 130 mL of methanol and stirred to dissolve to obtain solution 1. Phenylalanine (225.75 g, 1.0 eq) was added to 200 mL of methanol and stirred to obtain solution 2.
(2) The flow rates of solution 1 and solution 2 were controlled to be 1.0 mL/min and 1.2 mL/min, respectively. The temperature in the micromixer was 60° C., and the temperature in the microchannel reactor was 60° C. The residence time for the reaction mixture was 2.3 min.
(3) The reaction mixture flowing from the micro-reaction system was collected and subjected to concentration and separation and purification to obtain hydroxymethylphenylcarbamic acid with a yield of 73%.

### Example 6

Provided herein was a method for continuous preparation of amino alcohol compounds using a micro-reaction system. The method was basically the same as that of Example 4, except that the micromixer 3 used in this example was a Y-shaped micromixer. The target product isopropanolamine had a yield of 71%.

### Example 7

Provided herein was a method for continuous preparation of amino alcohol compounds using a micro-reaction system. The method was basically the same as that of Example 4, except that the micromixer 3 used in this example was a SX-type micromixer. The target product isopropanolamine had a yield of 62%.

### Example 8

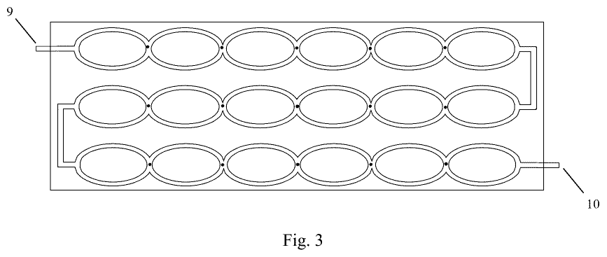

Provided herein was a method for continuous preparation of amino alcohol compounds using a micro-reaction system. The method was basically the same as that of Example 4, except that the microchannel reactor 4 was a gourd-shaped dispersion-convergence microchannel reactor. As shown in FIG. 3, the inner diameter of each of the inlet channel 9 and the outlet channel 10 was 800 μm, the long semiaxis of the external ellipse was 15 mm, the short semiaxis of the external ellipse was 9 mm, the long semiaxis of the internal ellipse was 13 mm, and the short semiaxis of the external ellipse was 7 mm. The center point of the crossing of adjacent ellipses was provided with a solid circle with a diameter of 0.5 mm for cutting fluid. The target product isopropanolamine had a yield of 85%.

What is claimed is:

1. A continuous-flow preparation method for an amino alcohol compound using a micro-reaction system, the micro-reaction system comprising a first feed pump, a second feed pump, a micromixer, a microchannel reactor and a back pressure valve, the micromixer, the microchannel reactor and the back pressure valve being connected in sequence, and the continuous-flow preparation method comprising:
(1) simultaneously feeding a solution of an aldehyde compound in a first solvent and a solution of an amine compound in a second solvent to the micromixer respectively through the first feed pump and the second feed pump for mixing to obtain a mixed
R¹—CHO
solution, wherein the aldehyde compound is represented by (II), and the amine compound is represented by 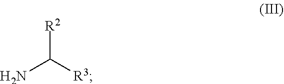
(2) feeding the mixed solution into the microchannel reactor to carry out an addition reaction to obtain a reaction mixture; and
(3) collecting the reaction mixture flowing out of the microchannel reactor, followed by concentration and separation purification to obtain the amino alcohol compound of formula (I): 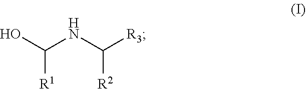
wherein R¹, R², and R³ are each independently selected from the group consisting of hydrogen, halogen, a C₁-C₁₂ alkyl, a C₃-C₆ cycloalkyl, a C₁-C₆ alkoxy, an unsaturated alkyl, an aromatic group, a nitrogen-containing alkyl, a sulfur-containing alkyl, a carboxyl group, an amide group, an aldehyde group, and an ester group.

2. The continuous-flow preparation method of claim 1, wherein in step (1), the first solvent and the second solvent are each independently selected from the group consisting of toluene, ethylbenzene, acetonitrile, n-butyronitrile, acetone, butanone, methyl isobutyl ketone, tetrahydrofuran, 2-methyltetrahydrofuran, 1,4-dioxane, dimethylsulfoxide (DMSO), dimethylformamide (DMF), methanol, ethanol, n-propanol, isopropanol, n-butanol, isobutanol, ethyl ether, methyl tert-butyl ether, and a combination thereof.

3. The continuous-flow preparation method of claim 1, wherein in step (1), a temperature in the micromixer is controlled at 25-60° C.; and flow rates of the solution of the aldehyde compound and the solution of the amine compound are controlled such that a molar ratio of the aldehyde compound to the amine compound in the micromixer is 1:0.5-6.

4. The continuous-flow preparation method of claim 1, wherein in step (2), a temperature in the microchannel reactor is controlled at 30-60° C.; and a residence time of the mixed solution in the microchannel reactor is 2-10 min.

5. The continuous-flow preparation method of claim 1, wherein in step (2), a back pressure of the back pressure valve is set to 0.1-2 MPa.

6. The continuous-flow preparation method of claim 1, wherein the micromixer is selected from the group consisting of a static mixer, a coaxial flow micromixer, a flow-focusing micromixer, a T-shaped mixer, a Y-shaped mixer, a Z-shaped mixer, a X-shaped mixer, and a divergent-convergent mixer.

7. The continuous-flow preparation method of claim 1, wherein the microchannel reactor is a tubular microchannel reactor or a plate-type microchannel reactor;
an inner diameter of the tubular microchannel reactor is 100 μm-8 mm; and
the plate-type microchannel reactor comprises a first heat exchange layer, a reaction layer, and a second heat exchange layer sequentially arranged from top to bottom; and the reaction layer is provided with a reaction fluid channel having a hydraulic diameter of 100 μm-8 mm.

8. The continuous-flow preparation method of claim 6, wherein the micromixer is a divergent-convergent mixer composed of a plurality of mixing units connected in series; each of the plurality of mixing unit comprises an outer square tube, an inner round tube, and an annular mixing channel arranged between the outer square tube and inner round tube; adjacent two mixing units among the plurality of mixing units are connected via a conduit at two opposite corners of outer square tubes of the adjacent two mixing units; and the mixed solution is divided in the annular mixing channel and converges at corners.

9. The continuous-flow preparation method of claim 7, wherein the microchannel reactor is a gourd-shaped divergent-convergent microchannel reactor; the microchannel reactor is composed of a plurality of circular or elliptical rings connected sequentially; adjacent two circular or elliptical rings among the plurality of circular or elliptical rings are communicated via conduits, or are tangent to each other and communicated at a tangent point; and the adjacent two circular or elliptical rings form a gourd-shaped structure, such that the reaction mixture is divided within each of the adjacent two circular or elliptical rings and converge at junctions between the adjacent two circular or elliptical rings.
